(19) United States
(12) Patent Application Publication
(10) Pub. No.: US 2008/0119757 A1
(43) Pub. Date: May 22, 2008

(54) Temperature management system with wireless patient temperature sensor

(76) Inventor: Suzanne Winter, Grass Valley, CA (US)

Correspondence Address: ROGITZ & ASSOCIATES, 750 B STREET, SUITE 3120, SAN DIEGO, CA, 92101, US

(21) Appl. No.: 11/602,635

(22) Filed: Nov. 21, 2006

(51) Int. Cl. A61B 5/01

(52) U.S. Cl. 600/549

(57) ABSTRACT

A system and method uses a wireless temperature sensor in a patient to provide feedback to a patient temperature control system for warming or cooling the patient using an external pad or indwelling catheter.

### FIELD OF THE INVENTION

The present invention relates generally to patient temperature control systems.

### BACKGROUND OF THE INVENTION

It has been discovered that the medical outcome for a patient suffering from severe brain trauma or from ischemia caused by stroke or heart attack or cardiac arrest is improved if the patient is cooled below normal body temperature (37° C.). Furthermore, it is also accepted that for such patients, it is important to prevent hyperthermia (fever) even if it is decided not to induce hypothermia. Moreover, in certain applications such as post-CABG surgery, it might be desirable to rewarm a hypothermic patient.

As recognized by the present invention, the above-mentioned advantages in regulating temperature can be realized by cooling or heating the patient's entire body. Moreover, the present invention understands that since many patients already are intubated with central venous catheters for other clinically approved purposes anyway such as drug delivery and blood monitoring, providing a central venous catheter that can also cool or heat the blood requires no additional surgical procedures for those patients. The following U.S. patents, all of which are incorporated herein by reference, disclose various intravascular catheters/systems/methods: U.S. Pat. Nos. 6,749,625, 6,786,916, 6,581,403, 6,454,792, 6,436,130, 6,146,411, 6,109,783, 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559, and U.S. patent application Ser. No. 10/355,776. Less optimally, surface cooling can be used. U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein by reference, disclose such surface cooling systems. In both intravascular catheters and external pad systems, working fluid such as a gas or saline is circulated through the heat exchange element.

Regardless of the particular heat exchange element that is engaged with the patient, as understood herein the above systems typically require temperature feedback from the patient. The feedback is provided by a temperature sensor engaged with the patient and connected to the catheter or pad controller using a wire. As understood herein, space is limited in most hospitals and adding another wire to the large number of wires already encumbering caregivers desirably is to be avoided.

### SUMMARY OF THE INVENTION

A method includes engaging a heat exchange element such as an external pad or an intravascular temperature control catheter with a patient. The heat exchange element is coupled to a control system for controlling patient temperature. A temperature sensing assembly is engaged with the patient, and the temperature sensing assembly includes a temperature sensor that generates a signal representative of patient temperature. Further, the temperature sensing assembly includes a wireless transmitter wirelessly transmitting a signal representative of patient temperature to the control system. The control system includes a wireless receiver receiving the signal from the transmitter, such that the control system controls, e.g., the temperature of fluid flowing through the heat exchange element at least partially based on the signal from the temperature sensing assembly.

Without limitation, the temperature sensing assembly can be embodied in a urethral catheter, or a bladder catheter, or a rectal catheter, or an esophageal catheter.

In another aspect, a system for exchanging heat with working fluid flowing through a patient-engageable heat exchange element includes a wireless receiver and a processor receiving data from the receiver. The processor executes logic to receive a target temperature signal and to use the target temperature signal and the data from the wireless receiver to establish, e.g., the temperature of the working fluid. In addition to or in lieu of temperature, the flow rate of the working fluid through the heat exchange element may be controlled.

In yet another aspect, a patient temperature control system includes a heat exchange element engageable with a patient's body and a controller controlling at least on parameter (such as temperature or flow rate) of working fluid circulating through the heat exchange element. Means are associated with the controller for receiving a target temperature. Also, means are associated with the controller for wirelessly receiving a patient temperature signal. As set forth further below, the patient temperature control system further includes means engageable with the patient for wirelessly transmitting the patient temperature signal to the means for wirelessly receiving on the controller. The controller uses the target temperature and patient temperature signal to control at least one parameter of the working fluid.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic diagram of a system according to present principles; and

FIG. 2 is a diagram showing a non-limiting wireless patient temperature sensor for use with a patient temperature control system.

### DETAILED DESCRIPTION OF THE PREFERRED EMBODIMENT

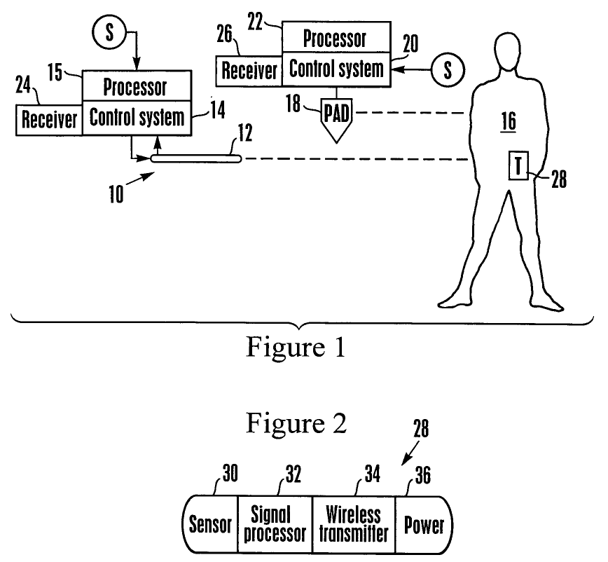

Referring initially to FIG. 1, a system is shown, generally designated 10, that may include a heat exchange catheter 12 that is in fluid communication with a catheter temperature controller 14 that includes a processor 15 executing logic described in one or more of the patents referenced herein for controlling the temperature of working fluid flowing through the catheter 12.

In accordance with present principles, the system 10 can be used to induce therapeutic hypothermia in a patient 16 using a catheter in which working fluid such as but not limited to saline circulates in a closed loop, such that no working fluid enters the body. While certain preferred catheters are disclosed below, it is to be understood that other catheters can be used in accordance with present principles, including, without limitation, any of the catheters disclosed in the following U.S. patents, all incorporated herein by reference: U.S. Pat. Nos. 5,486,208, 5,837,003, 6,110,168, 6,149,673, 6,149,676, 6,231,594, 6,264,679, 6,306,161, 6,235,048, 6,238,428, 6,245,095, 6,251,129, 6,251,130, 6,254,626, 6,261,312, 6,312,452, 6,325,818, 6,409,747, 6,368,304, 6,338,727, 6,299,599, 6,287,326, 6,126,684. The catheter 12 may be placed in the venous system, e.g., in the superior or inferior vena cava without blocking the vessel so that blood can flow around the catheter to effect heat exchange. Examples of non-limiting controllers 14 are shown in U.S. Pat. Nos. 6,786,916, 6,581,403, 6,454,792, 6,436,130, 6,146,411, and 6,109,783, incorporated herein by reference.

Instead of or in addition to the catheter 12, the system 10 may include one or more pads 18 that are positioned against the external skin of the patient 16 (only one pad 18 shown for clarity). The pad 18 may be, without limitation, any one of the pads disclosed in the external pad patents. The temperature of the pad 18 can be controlled by a pad controller 20 with processor 22 in accordance with principles set forth in the external pad patents to exchange heat with the patient 16, including to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia. The controllers 14, 20 may be implemented by a single system having one or more processors for executing temperature control algorithms in accordance with the referenced patents. Instead of inducing therapeutic hypothermia the catheter 12/pad 18 may be used to prevent fever, i.e., to maintain normothermia, and/or to rewarm a cold patient.

In any case, as shown in FIG. 1 a controller 14, 20 of the present invention includes means “S” for entering a target patient temperature. To this end, a knob or keypad or other data entry device may be mounted on the console of the controller. Furthermore, a controller 14, 20 of the present invention includes means for wirelessly receiving patient temperature signals from the patient. The receiving means can include wireless receivers 24, 26, respectively shown as being integrated with the controllers 14, 20.

Additionally, a temperature sensing assembly 28 is engaged with the patient 16 as shown. FIG. 2 shows that the assembly 28 includes a temperature sensor 30 such as but not limited to a thermocouple, resistance temperature detector (RTD), etc. that generates a signal representative of patient temperature and that sends the signal to signal processing circuitry 32. In non-limiting implementations the circuitry 32 may include signal conditioning and amplification circuitry, as well as analog-to-digital conversion circuitry and digital processing circuitry if desired.

As contemplated herein, the assembly 28 includes a wireless transmitter 34 for wirelessly transmitting a signal derived from the sensor 30 signal to the wireless receiver or receivers 24, 26 shown in FIG. 1. The assembly 28 may be energized by a power source 36 such as a small battery.

It may now be appreciated that the wireless transmitter 34 uses the same wireless communication principles as are used by the receivers 24, 26. In some embodiments short range radiofrequency (RF) principles may be used. Some short range RF protocols that can be used are colloquially referred to as “Bluetooth.” Wireless 802.11 communication principles may be used. Instead of RF, ultrasonic communication principles alternatively may be used. Indeed, in some implementations near field communications (NFC) principles may be used, in which case the transmitter 34 may be a passive radiofrequency identification (RFID) token that is read by the receiver or receivers 24, 26, which can be implemented by RFID readers. When RFID is used the power source 36 can be dispensed with.

The assembly 28 may be embodied, without limitation, in a urethral catheter such as a so-called “Foley” catheter, or in a bladder catheter, or in a rectal catheter, or in an esophageal catheter, or by a tympanic temperature sensing apparatus. Skin-mounted assemblies less optimally may be used.

While the particular TEMPERATURE MANAGEMENT SYSTEM WITH WIRELESS PATIENT TEMPERATURE SENSOR is herein shown and described in detail, it is to be understood that the subject matter which is encompassed by the present invention is limited only by the claims.

What is claimed is:

1. A method comprising:
engaging a heat exchange element with a patient, the heat exchange element being coupled to a controller for establishing patient temperature;
engaging a temperature sensing assembly with the patient, the temperature sensing assembly including a temperature sensor generating a signal representative of patient temperature, the temperature sensing assembly including a wireless transmitter wirelessly transmitting a signal representative of patient temperature to the controller, the controller including a wireless receiver receiving the signal from the transmitter, the controller controlling at least the temperature of fluid flowing through the heat exchange element at least partially based on the signal from the temperature sensing assembly.

2. The method of claim 1, wherein the heat exchange element includes an intravascular catheter.

3. The method of claim 1, wherein the heat exchange element includes at least one pad applied externally to a patient's skin.

4. The method of claim 1, wherein the temperature sensing assembly is embodied in a urethral catheter.

5. The method of claim 1, wherein the temperature sensing assembly is embodied in an esophageal catheter.

6. The method of claim 1, wherein the temperature sensing assembly is embodied in a rectal catheter.

7. The method of claim 1, wherein the temperature sensing assembly is embodied in a bladder catheter.

8. A control system for exchanging heat with working fluid flowing through a patient-engageable heat exchange element, comprising:
a wireless receiver;
a processor receiving data from the receiver and executing logic comprising:
receiving a target temperature signal; and
using the target temperature signal and the data from the wireless receiver to establish at least the temperature of the working fluid.

9. The control system of claim 8, comprising:
a temperature sensing assembly engageable with the patient, the temperature sensing assembly including:
a temperature sensor generating a signal representative of patient temperature; and
a wireless transmitter receiving signals from the sensor and wirelessly transmitting a signal representative of patient temperature to the control system.

10. The control system of claim 8, wherein the heat exchange element includes an intravascular catheter.

11. The control system of claim 8, wherein the heat exchange element includes at least one pad applied externally to a patient's skin.

12. The control system of claim 9, wherein the temperature sensing assembly is embodied in a urethral catheter.

13. The control system of claim 9, wherein the temperature sensing assembly is embodied in an esophageal catheter.

14. The control system of claim 9, wherein the temperature sensing assembly is embodied in a rectal catheter.

15. The control system of claim 9, wherein the temperature sensing assembly is embodied in a bladder catheter.

16. A patient temperature controller, comprising:
at least one heat exchange element engageable with a patient's body;
at least one controller controlling at least one parameter of working fluid circulating through the heat exchange element;
means associated with the controller for receiving a target temperature;
means associated with the controller for wirelessly receiving a patient temperature signal; and
means engageable with the patient for wirelessly transmitting the patient temperature signal to the means for wirelessly receiving, wherein
the controller uses the target temperature and patient temperature signal to control at least one parameter of the working fluid.

17. The controller of claim 16, wherein the means for wirelessly transmitting is embodied by a urethral catheter or a bladder catheter.

18. The controller of claim 16, wherein the means for wirelessly transmitting is embodied by an esophageal catheter.

19. The controller of claim 16, wherein the means for wirelessly transmitting is embodied by a rectal catheter.

20. The controller of claim 16, wherein the heat exchange element includes an intravascular catheter.
